(19) United States
(12) Patent Application Publication
(10) Pub. No.: US 2019/0247143 A1
(43) Pub. Date: Aug. 15, 2019

(54) Marker Assembly for Use in Clinical Marking

(71) Applicant: Guy's and St. Thomas' NHS Foundation Trust, London (GB)

(72) Inventors: Andrew John Coleman, London (GB); Richard Smith, Gamlingay (GB)

(21) Appl. No.: 16/396,061

(22) Filed: Apr. 26, 2019

Related U.S. Application Data
(63) Continuation of application No. PCT/GB2017/053219, filed on Oct. 25, 2017.

(30) Foreign Application Priority Data
Oct. 26, 2016 (GB) 1618119.0

(51) Int. Cl. A61B 90/00; B43K 23/12; B43K 8/03; B43K 8/02; B43K 1/12

(52) U.S. Cl. CPC A61B 90/39; B43K 23/12; A61B 2090/395; B43K 8/022; B43K 1/12; B43K 8/03

(57) ABSTRACT

A marking implement assembly for placing a mark on tissue, including a marker body including a marking implement extending from the marker body, a cap configured to fit to an end of the marker body so as to cover the marking implement, the cap including a housing providing an ink reservoir, where when fitted to the marker body at least a part of the marking implement is in fluid contact with the ink reservoir, the cap fitting onto the marker body in fluid tight manner.

### CROSS-REFERENCE TO RELATED APPLICATIONS

This application is a continuation of International Application No. PCT/GB2017/053219 having an international filing date of Oct. 25, 2017 entitled “Marker Assembly for Use in Clinical Marking”. The '219 international application claimed priority benefits, in turn, from United Kingdom Patent Application No. 1618119.0 filed on Oct. 26, 2016.

The '219 international application and the '119.0 UK application are hereby incorporated by reference herein in their entireties.

### FIELD OF THE INVENTION

The present invention relates to a marker assembly for placing a mark on tissue, as well as to a marker tool for placing a mark on tissue. The marker assembly is particularly useful for ensuring the marker is ready for use. Another aspect of the invention also provides for fitting a marker body to a marking instrument.

A number of clinical procedures require marking of tissue, in particular marking an area of clinical interest on the surface of the skin. In such procedures, a surgeon or operator applies marks directly on the surface of the skin using a handheld felt tip marker pen. These pens are normally sterile and individually packaged. They typically use a surgical grade ink, often a formulation of gentian violet.

In order to prevent cross-contamination between patients, the pens can only be used for a single procedure performed on a single patient.

Marking of tissue can also be done using a handheld marking instrument to which a nib is attached. For best practice, the nibs should be replaced between patients.

### SUMMARY OF INVENTION

The present disclosure seeks to provide an improved marker implement assembly for placing a mark on tissue. In the preferred embodiment, there is provided an assembly for fitting and removing nibs from a marking instrument and for ensuring adequate ink provision in the nib.

According to an aspect of the present invention, there is provided a marking implement assembly for placing a mark on tissue, including: a marker body, a marking implement fitted to and extending from the marker body; a cap configured to fit to an end of the marker body so as to cover the marking implement, the cap including a housing providing an ink reservoir, wherein when fitted to the marker body at least a part of the marking implement is in fluid contact with the ink reservoir, the cap fitting onto the marker body in fluid tight manner; wherein said end of the marker body includes a first connector element integral with the marker body, and the cap includes a second connector element integral with the cap; wherein the first and second connector elements are inter-engageable in non-rotatable manner such that the marker body and the cap do not rotate relative to one another.

The feature of providing an ink reservoir in the cap ensures that the marker implement is always loaded with ink and in the preferred embodiments can be reloaded therefrom. The fluid tight fitting prevents ink escape and drying out of the ink.

The non-rotatable nature of the coupling between the marker implement and the cap allows the cap to be used to manipulate the marker body, for instance to fit this to an instrument as described below, without having to touch or handle the marker body itself.

In an embodiment, the ink reservoir includes an ink loaded wad.

The first and second connector elements may comprise a polygonal head and a corresponding polygonal recess, such as a hexagonal head and a corresponding hexagonal recess.

Advantageously, the cap is a push fit onto the marker body. In other embodiments, there may be a locking engagement of the cap to the marker body, such as by a screw thread or bayonet fitting.

In the preferred embodiment, the cap includes a hand grip. The hand grip may be substantially flat and extend laterally beyond the housing.

In preferred embodiments, the marker body includes a spring element operatively connected to the marking implement for absorbing pressure on the marking implement. The spring element may include a sponge or elastomeric material.

In practical embodiments, the marking implement has an absorbency to go from a dry to a usable inked condition within 30 seconds, preferably within 20 seconds, more preferably within 10 seconds.

Preferably, the marking implement is a nib, which may be of felt. Felt may have a natural resilience, which can make the need for a sprung connection unnecessary.

Advantageously, the marker assembly is a handheld device.

According to another aspect of the present invention, there is provided a marker assembly for placing a mark on tissue, including: a marker body including a first end with a first coupling element and a second end including a marking implement for marking tissue; a marking instrument including a second coupling element, wherein the first and second coupling elements interlock so as to connect the marker body to the marking instrument; a cap connectable onto the second end of the marker body, so as to cover the marking implement, leaving the first coupling element of the marker body exposed for attachment to the marking instrument, the cap being removable from the marker body so as to expose the marking implement.

Preferably, the first coupling element includes a screw thread or a bayonet fitting.

Advantageously, the second end of the marker body includes a first connector element and the cap includes a second connector element, wherein the first and second connector elements are inter-engageable in non-rotatable manner.

The cap may include a hand grip, that may be substantially flat and extend laterally beyond the housing.

In some embodiments, the cap includes a test element for testing correct fitting of the marker body to the marking instrument, the test element including a perimeter feature and a positional member, the perimeter feature being alignable with a body member of the marking instrument, whereby correct fitting results in the marker implement aligning with the positional member.

The positional member may be a pinpoint, preferably through which the tip of the marker implement can pass.

The perimeter feature may be a recess.

Advantageously, the test element is incorporated in the or a hand grip.

In some embodiments, the first coupling element includes a bayonet fitting

However, the first coupling element may include any other form of positive location fitment known to one of skill in the art, for interlocking with the second coupling element.

The tool disclosed herein therefore provides a complete assembly for locking and removing the marker body from the marking instrument, as well as for testing correct fitting of the marker body with the marking instrument

Other features and advantages of the assembly and tool disclosed herein will become apparent from the specific description which follows.

### BRIEF DESCRIPTION OF THE DRAWINGS

Embodiments of the present invention are described below, by way of example only, with reference to the accompanying drawings, in which:

FIG. 1 is a rear perspective view of an embodiment of marker assembly and cap as herein described;

FIG. 2 is a cross-sectional front perspective view of the marker assembly and the cap;

FIG. 3 is a front perspective view of the marker assembly and the cap assembled together;

FIG. 4 is a view in side elevation of the marking assembly and the cap assembled together;

FIG. 5 is a cross-sectional front perspective view of another embodiment of marking assembly and cap as herein described;

FIG. 6 is a front perspective view of the marking assembly and the cap assembled together;

FIG. 7 is an exploded view of the marker assembly and the cap;

FIG. 8 is a view in side elevation of the marking assembly and the cap assembled together;

FIG. 9 is a front perspective view of another embodiment of marking assembly as herein described;

FIG. 10 is a cross-sectional side view of the marker assembly of FIG. 9; and

FIG. 11 is a view in side elevation of the marking assembly of FIG. 9.

### DETAILED DESCRIPTION OF ILLUSTRATED EMBODIMENT(S)

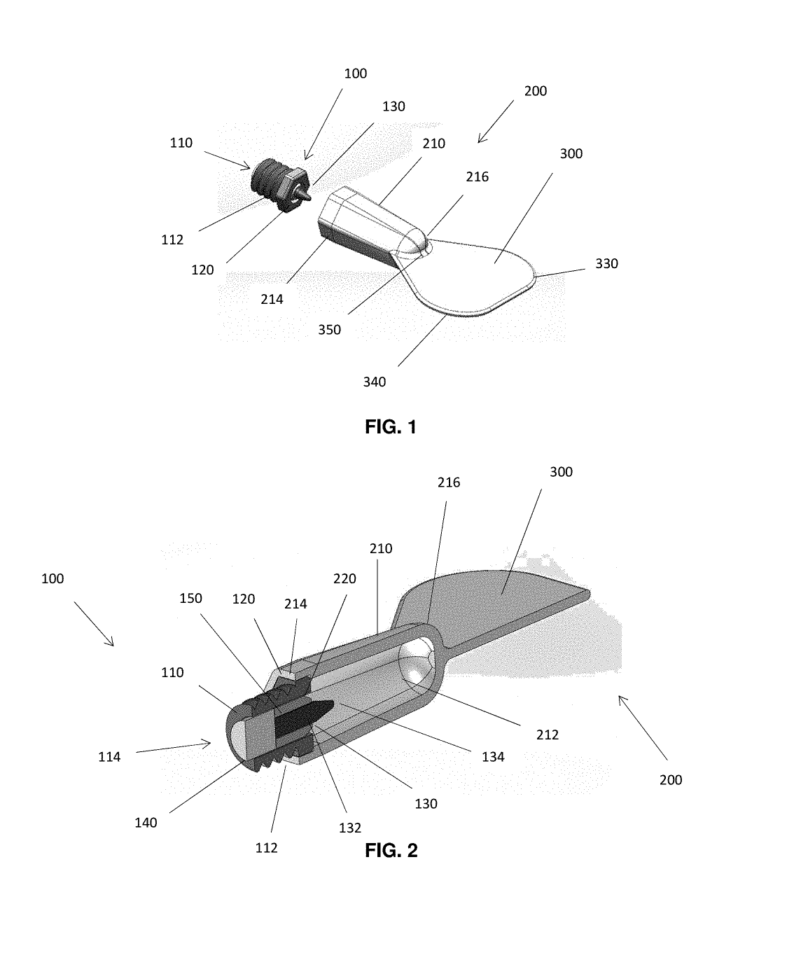

Described Referring to FIG. 1, the embodiment of marker assembly shown has a marker body 100, including a housing 110 of generally cylindrical form with a first end having an outer screw thread 112 and a second end with an enlarged hexagonal head 120. The marker assembly 100 also includes a marking implement 130 that is fitted into the marker body 100 and protrudes from the hexagonal head 120.

A cap 200 includes a housing 210 with a hexagonal opening 214 at one end and is closed at the other end 216. In this embodiment, the closed end 216 is shaped substantially as a hemisphere. The cap 200 includes a hand grip 300 integral with the housing 210. The hand grip 300 is substantially flat and extends laterally beyond housing 210 to provide sufficient gripping surface to enable a person to apply torque to the cap and hence the marker body 100. In the embodiment of FIG. 1, the hand grip 300 has a substantially triangular shape with rounded corners 330, 340. The third corner 350 is merged into end 216 of housing 210. In a preferred embodiment, the hand grip 300 and hexagonal housing 210 are formed as a single piece.

The cap 200 is preferably made of a plastics material, such as plastics resin or polyester. The skilled person will appreciate that there is a wide choice of suitable materials for the cap 200.

FIG. 2 shows a cross-sectional view of the marker assembly 100 and cap 200. The housing 112 includes an internal bore 114, which in this embodiment is circular in transverse cross-section and cylindrical.

In this embodiment, hexagonal head 120 is diametrically larger than threaded housing 112, however in other embodiments, it may be the same or smaller.

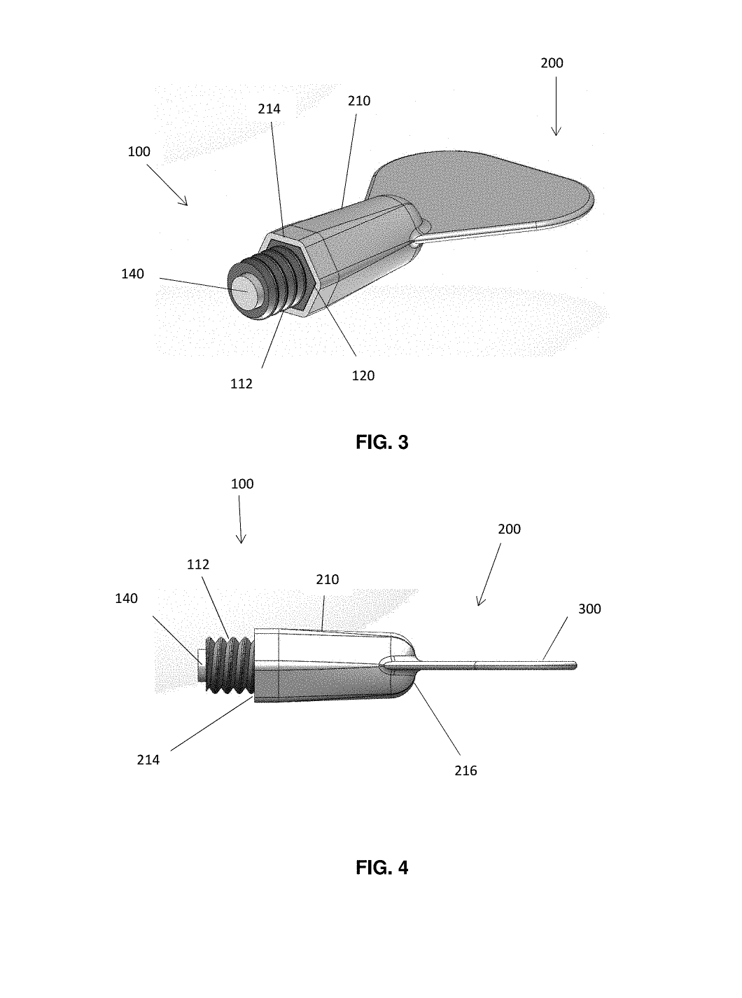

A spring 140 is shown, illustrated in diagrammatic form, fitted within the bore 114. In some embodiments, the spring 140 may be made of sponge or elastomeric material, or may be of a metallic or plastics material. In some embodiments, the body 120 includes a spring chamber which may protrude out from housing 112. This can be seen in FIGS. 3 and 4. The spring 140 extends approximately half the length of bore 114 but in other embodiments may be longer or shorter. The skilled person will appreciate that the sponge or spring will in practice not be exposed but will abut a sealing base plate or wall (not shown) of the body 110.

The marking implement 130, hereinafter referred to as nib, includes a cylindrical flat bottomed section 132 and a pointed conical tip 134. The cylindrical section 132 fits slidably within a sleeve 150 disposed in the bore 114 of the housing 112. Alternatively or additionally, the sleeve 150 is slidable within the bore 114 of the housing 112 and the nib 130 is fixed thereto. The base of the cylindrical section 132 abuts against the spring 140.

The nib 130 may be made of a porous or fibrous material, such as felt. In an exemplary embodiment, the nib 130 may be in the region of 4 mm in length and cylindrical section 132 may have a diameter of substantially 0.8 mm. In other embodiments, the nib 130 has the same dimensions as a standard nib of known surgical marking devices. Suitable inks for this purpose are known in the art and include inks that are washable, biocompatible, easily visible, quick drying and so on.

The hexagonal head 120 fits within hexagonal recess 214 of the cap 200 and is preferably a friction fit to hold the marker assembly 100 and cap 200 securely together and provide an airtight seal. This is the currently preferred arrangement, although it is not excluded that there may be an interlocking arrangement, such as a bayonet fitting or screw thread fitting (typically of the opposite hand to the screw thread of the housing 112).

The open recess 214 of the cap 200 includes an internal width restriction 220 which acts as a stop shoulder. In practice, the hexagonal head 120 abuts against the stop shoulder 220, preventing the marker body 110 from being inserted too far into the housing 210.

The cross-sectional view of FIG. 2 shows the nib tip 134 extending into the chamber 212 of the housing 210.

FIGS. 3 and 4 show views of the marker assembly 100 and cap 200 assembled together. As described above, the hexagonal head 120 fits within the hexagonal opening 214 of housing 210. The marker assembly 100 and cap 200 are connected such that the second end of the body 110 with the threaded surface is disposed outside of housing 210.

It will be seen that, in this embodiment, the casing 210 tapers slightly from its open front end 214 to the closed end 116.

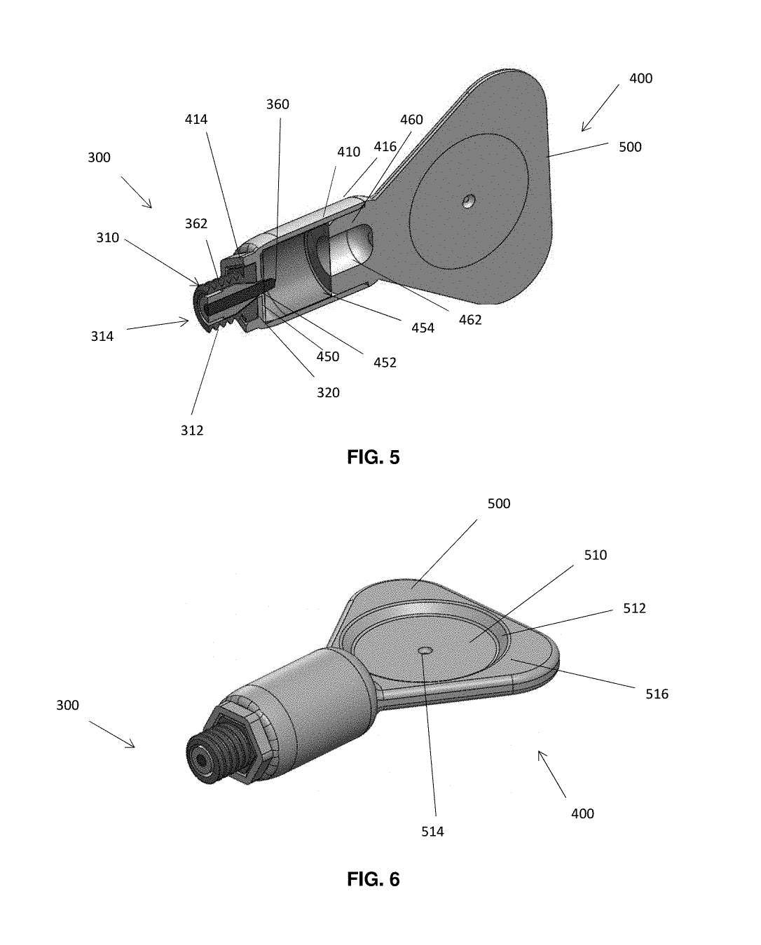

Referring to FIG. 5, this is another embodiment of marker assembly 300 and cap 400. The housing 410 of the cap 400 is of generally cylindrical form and includes a transverse panel or wall 450 near opening 414, against which the hexagonal head 320 of the marker housing 310 abuts. An aperture 452 in the wall 450 allows the nib 360 to extend through the wall 450 and into the housing 410. The nib 360 is disposed, in this embodiment, in a sleeve 362 with a tapering distal end. Not shown in the drawing but located at 314 is an end panel integral with the housing 310 that seals the proximal end of the housing 310. The housing 310 has an external screw thread 312.

The cap 400 includes an ink reservoir 454, which may be a hollow chamber or a wad of material. Preferably, the wad is circular in transverse cross-section and cylindrical, but may have other shapes. The reservoir 454 extends the length of the housing 410 but may be shorter. As can be seen in FIG. 5, the tip of the nib 360 extends into the ink reservoir 454 when the body 310 is fitted to the cap 400.

The ink reservoir 454 may be a wad of porous or fibrous material.

In the embodiment of FIG. 5, the cap 400 includes a hand grip 500 and a housing 410 as two separate components that are attached together in use. This enables easier manufacture of the cap assembly 400. The hand grip 500 includes a cylindrical element 460 that fits into an end 416 of the housing 410 and is a tight fit. In some embodiments, the hand grip 500 and the housing 410 are bonded or welded together following assembly. In the embodiment of FIG. 5, the element 460 includes a hollowed section 462 to reduce material bulk and increase efficiency in manufacturing.

In other embodiments, the hand grip 500 and housing 410 are formed as a single piece.

FIG. 6 shows a perspective view of the assembly of FIG. 5, where it can be seen that the hand grip 500 includes a test element 510. The test element 510 includes a perimeter feature 512 which in the embodiment of FIG. 6 is a bevelled or tapering peripheral wall of a circular recess. A positional member 514, formed as a circular pinpoint or aperture, is located at the centre of the perimeter element 512.

In other embodiments, the perimeter element 512 may be any other suitable shape.

While FIG. 6 depicts the marker assembly 300 as having an open proximal end, as explained above, this will typically have an end panel or wall to seal it closed.

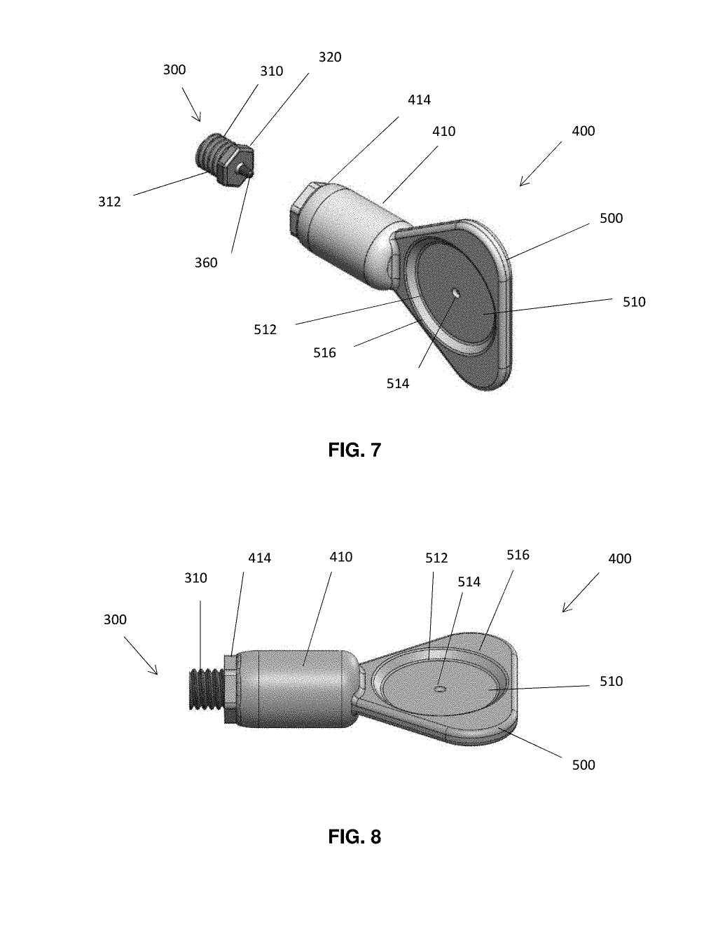

FIG. 7 shows the marker assembly 300 removed from the cap 400, while FIG. 8 shows another view of the marker assembly 300 and the cap 400 when connected together.

In the embodiment of FIGS. 5 to 8, there is no spring device behind the nib 360. This is an optional feature.

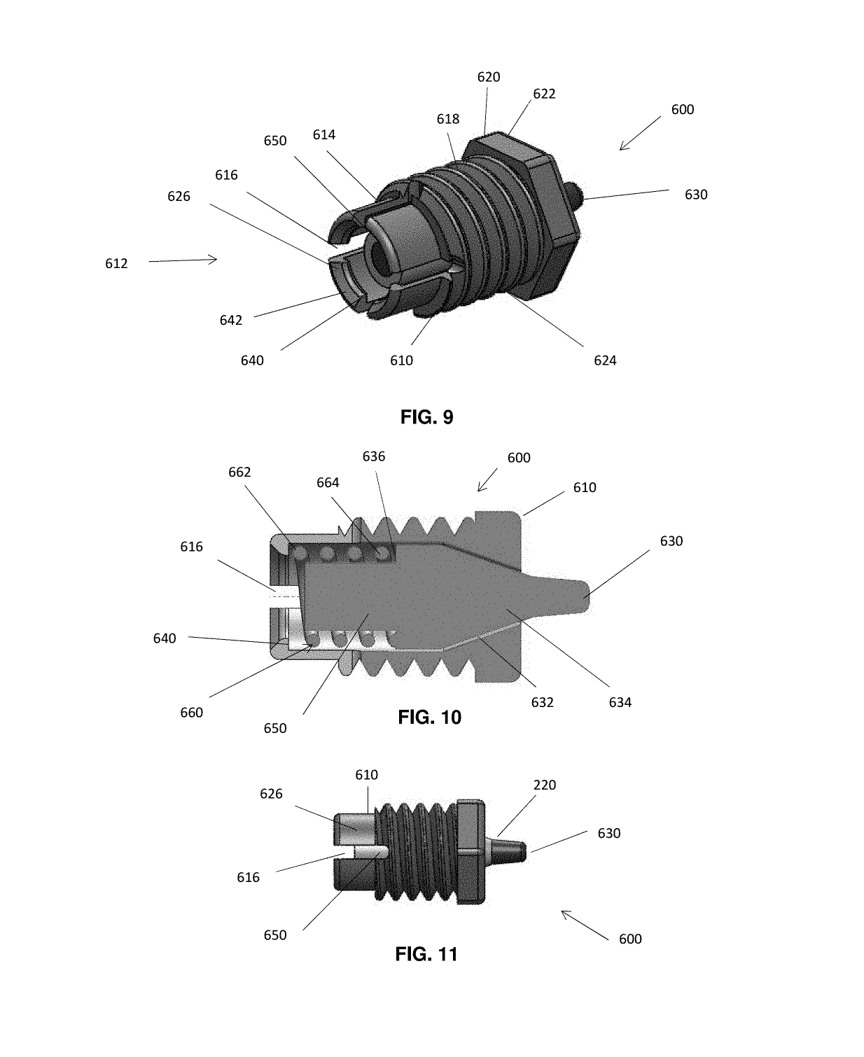

FIGS. 9 to 11 show another embodiment of a marker assembly body 600, including a housing 610 of generally cylindrical form with an internal bore that is circular in transverse cross-section. Housing 610 includes a first end 614 including a plurality of radially spaced slits 616 extending through a portion of the housing 610, a second end 620 with an enlarged hexagonal head 622 and an intermediate portion 618 disposed between the first end 614 and the second end 620 of the housing 610 and including an outer screw thread 624. The slits 616 separate the first end 614 of the housing 610 into a series of circumferentially disposed resilient legs 626.

A nib 630 can be seen protruding from the hexagonal head 622. FIG. 9 shows four slits and corresponding legs, but in other embodiments fewer or more slits and legs may be provided. At the first end 614 of the housing 610, flanges 640 project internally from the edge 642 of the housing 610, that is the legs, towards the centre of the housing 610.

The nib 630 includes a cylindrical proximal section 650 disposed within the bore 612 of housing 610.

Referring to FIG. 10, a spring 660 is shown fitted within the bore 612 of the housing 610. A first end 662 of the spring 660 abuts against the flanges 640 which act as stop shoulders.

The cylindrical section 650 of the nib 630 fits within the spring 660 and has a radially larger shoulder 636 that abuts against a second end 664 of the spring 660. The spring 660 is therefore trapped between the flanges 640 and the shoulder 636 and in practice acts to bias the nib 630 forwards. The nib 630 has a tapering distal end 634 that fits within a tapering section 632 of the housing 610, such that the nib 630 is held securely in the housing, although able to move into the housing against the spring bias when the nib 630 is pressed.

The distal taper 634 of the nib 630 also enables the nib 630 into the housing 610 from the proximal end, that is passed the legs 262, which will splay outwardly to allow passage of the nib 630. The spring 660 can be interested in similar manner into the housing 610, after fitting of the nib 630, which will then become trapped as the legs 626 spring back to their rest position.

In practice, as the nib and cap assembly would be stored in an airtight pouch, the nib and ink reservoir will not dry out before use.

In use, the nib 130 is loaded with ink from the ink reservoir in the cap. Preferably the nib 130 holds a sufficient amount of ink to mark an area of interest. In a preferred embodiment, the nib 130 holds sufficient ink to apply a 3 cm long mark on skin, although in other embodiments it may hold sufficient ink to a greater marking length. In the configuration shown in FIGS. 2 to 6 and 8, which can be done even when the body 110 is attached to another instrument. The ink reservoir in the cap may hold substantially more ink such that the nib can be reloaded. This can be achieved by replacing the cap onto the nib to allow the nib to soak up further ink from the reservoir.

Referring again to FIGS. 2 and 5 in particular, the cap seals the nib 130 in fluid tight manner, preventing the ink in the nib 130 from drying out.

The marker assembly 100 is designed to be fitted to a marking instrument (not shown). The threaded housing 120 is screwed into a threaded bore in a structure of a marking instrument. The marking instrument may be a pen barrel, an element of an imaging device and so on.

The hand grips 300, 500 provide a relatively large and wide surface area to aid gripping, for example between a finger and a thumb. This helps with ease of handling the marker assembly 100 and cap 200, together as well as to apply torque to the housing 120. Once the threaded housing 120 is firmly screwed into a marking instrument, the cap 200 can be removed by pulling away from the marker assembly 100. The nib 130 is then exposed for use.

After use, marker assembly 100 can be detached from the marking instrument, using the cap 200 to remove it and replace after use. It will be appreciated that the used nib will then be safely housed in the cap, preventing contamination.

The marker assembly 100 and cap 200 are supplied packaged and assembled together. This ensures the nib 160 is kept in constant fluid contact with the ink reservoir 254 so as to remain loaded with ink and ready for use. In the event that a marking procedure is prolonged and further ink is required, the cap 200 can be fitted back onto marker assembly 100. As hexagonal head 120 fitted into hexagonal opening 214, the marking implement 160 is pushed into ink reservoir 254. The nib 160 preferably has an absorbency which enables it to become fully restored with ink from a dry condition in around 10 seconds. This provides an easy and convenient way of replenishing marking implement 160 with ink, without the operator having to fiddle with replacing the whole marker assembly 100 with a new one.

Referring again to FIGS. 6 to 8, the test feature 510 on hand grip 500 enables an operator to check readily whether the marker body 300 has been correctly fitted to a marking instrument. To test this, the operator brings perimeter element 512 to a corresponding feature on the marking instrument and aligns the two together. In the embodiment of FIGS. 5 to 8, the perimeter element 512 is aligned with a cylindrical body or a body of circular cross-section protruding from the marking instrument. The bevelled edge 516 helps align the perimeter element 512 to the body of a marking instrument. Test area 510 is therefore accurately located with respect to marking instrument. If the marker assembly 300 has been fitted correctly, the nib will align with the pinpoint 514.

As will be apparent, the marker body 100, 300, 600 is effectively provided with two coupling devices, the first being the coupling to the cap, which in the preferred embodiments is a non-rotatable fit such as of polygonal shape; while the second is the coupling for attachment to a marker system, which in the embodiments shown in the threaded coupling but may have other forms such as a bayonet fitting and so on. The double coupling enables the marker implement to be attached to a cap that can then be used for positioning and applying rotational and/or compressing or tensile force, and also attached securely to a marker system while the marker implement is fitted to and held by the cap. Similarly, the cap can be used to remove the marker implement from the system after use.

The marker assembly 100, 300 and the cap 200, 500 may come in multiples packaged in a blister pack, for example as a set of 5 or 10 in each pack.

All optional and preferred features and modifications of the described embodiments and dependent claims are usable in all aspects of the invention taught herein. Furthermore, the individual features of the dependent claims, as well as all optional and preferred features and modifications of the described embodiments are combinable and interchangeable with one another.

What is claimed is:

1. A marking implement assembly for placing a mark on tissue, including:
a marker body,
a marking implement fitted to and extending from the marker body;
a cap configured to fit to an end of the marker body so as to cover the marking implement, the cap including a housing providing an ink reservoir, wherein when fitted to the marker body at least a part of the marking implement is in fluid contact with the ink reservoir, the cap fitting onto the marker body in fluid tight manner;
wherein said end of the marker body includes a first connector element integral with the marker body, and the cap includes a second connector element integral with the cap; wherein the first and second connector elements are inter-engageable in non-rotatable manner such that the marker body and the cap do not rotate relative to one another;
wherein the cap is removable to expose the marking implement for use.

2. A marker assembly according to claim 1, wherein the ink reservoir includes an ink loaded wad.

3. A marker assembly according to claim 1, wherein the first and second connector elements comprise a polygonal head and a corresponding polygonal recess.

4. A marker assembly according to claim 1, wherein the first and second connector elements comprise a hexagonal head and a corresponding hexagonal recess.

5. A marker assembly according to claim 1, wherein the cap is a push fit onto the marker body.

6. A marker assembly according to claim 1, wherein the cap includes a hand grip.

7. A marker assembly according to claim 6, wherein the hand grip is substantially flat and extends laterally beyond the housing.

8. A marker assembly according to claim 1, wherein the marker body includes a spring element operatively connected to the marking implement for absorbing pressure on the marking implement.

9. A marker assembly according to claim 8, wherein the spring element includes a sponge or elastomeric material.

10. A marker assembly according to claim 1, wherein the marking implement has an absorbency to go from a dry to a usable inked condition within 30 seconds, preferably within 20 seconds, more preferably within 10 seconds.

11. A marker assembly according to claim 1, wherein the marking implement is a nib.

12. A marker assembly according to claim 1, wherein the nib is of felt.

13. A marker assembly according to claim 1, wherein the marker assembly is a handheld device.

14. A marker assembly for placing a mark on tissue, including:
a marker body including a first end with a first coupling element and a second end including a marking implement for marking tissue;
a marking instrument including a second coupling element, wherein the first and second coupling elements interlock so as to connect the marker body to the marking instrument;
a cap connectable onto the second end of the marker body, so as to cover the marking implement, leaving the first coupling element of the marker body exposed for attachment to the marking instrument, the cap being removable from the marker body so as to expose the marking implement.

15. A marker assembly according to claim 14, wherein the first coupling element includes a screw thread or a bayonet fitting.

16. A marker assembly according to claim 14, wherein the second end of the marker body includes a first connector element and the cap includes a second connector element, wherein the first and second connector elements are inter-engageable in non-rotatable manner.

17. A marker assembly according to claim 14, wherein the cap includes a hand grip.

18. A marker assembly according to claim 14, wherein the cap includes a test element for testing correct fitting of the marker body to the marking instrument, the test element including a perimeter feature and a positional member, the perimeter feature being alignable with a body member of the marking instrument, whereby correct fitting results in the marker implement aligning with the positional member.

19. A marker assembly according to claim 18, wherein the positional member is a pinpoint.

20. A marker assembly according to claim 18, wherein the perimeter feature is a recess.
